(19) United States
(12) Patent Application Publication
(10) Pub. No.: US 2013/0131763 A1
(43) Pub. Date: May 23, 2013

(54) METHOD FOR MOUNTING A WEARABLE HORSE COOLING DEVICE

(71) Applicants: Gary CHIU, Plano, TX (US); Naijian Fu, Fuzhou (CN)

(72) Inventors: Gary CHIU, Plano, TX (US); Naijian Fu, Fuzhou (CN)

(21) Appl. No.: 13/299,815

(22) Filed: Nov. 18, 2011

(51) Int. Cl. A61F 7/10; A61D 99/00

(52) U.S. Cl. 607/104

(57) ABSTRACT

A method for mounting a wearable horse cooling device performed by a wearable horse cooling device having two cold water circulating modules and a fixing module, wherein each cold water circulating module has a cooling unit, a wrap and a connection hose assembly, has steps of placing the cooling unit of each cold water circulating module on a body of a horse, adjusting the fixing module and securely connecting the fixing module with each cold water circulating module, connecting the connection hose assembly with the cooling unit and the wrap of each cold water circulating module and mounting the wrap of each cold water circulating module around a portion of the body of the horse, and setting operation parameters for a cooling cycle. Accordingly, the method allows a horse carrying the wearable horse cooling device to freely move during a cooling cycle.

### BACKGROUND OF THE INVENTION

1. Field of the Invention

The present invention relates to a method for mounting a wearable horse cooling device, and more particularly to a method for mounting a wearable horse cooling device to lower the temperature of and apply cold compresses to a horse.

2. Description of the Related Art

Horse cooling devices are more commonplace in western world and are available to lower the temperature of and apply cold compresses to horses, especially useful in applying cold compresses as horses usually exercise strenuously and easily get hurt.

The principle of a horse cooling device is that low temperature cold water in a container is pumped into a water inlet of a water bag by a water pump and is circulated back to the container through a water outlet of the water bag. Thus, outside surfaces of the water bag can be kept at low temperature. The water bag is attached to a portion of the body of a horse requiring a temperature drop or a cold compress and is strapped and fastened on the body of the horse to constantly lower temperature of or apply cold compresses to the horse.

Conventional horse cooling devices tend to be bulky and heavy, need to be placed beside horses, and are connected to the cooling water bags fastened on the bodies of horses through water pipes and air pipes. When such a horse cooling device is applied to cool temperature of or apply a cold compress to a horse, the horse must be confined in a tiny space to prevent the horse from running around. Otherwise, wild horse movement can break off the water pipes and air pipes connected between a cooling unit and the water bag and even pull and damage the cooling unit and the water bag. Moreover, due to prolonged confinement in a tiny space and limited movement, horses easily get irritable and fiercely kick and jump at the cost of horses' health and smoothness of cold compress process.

### SUMMARY OF THE INVENTION

An objective of the present invention is to provide a method for mounting a wearable horse cooling device to lower the temperature of and apply cold compresses to a horse.

To achieve the foregoing objective, the method for mounting a wearable horse cooling device is performed by a wearable horse cooling device having at least one cold water circulating module and a fixing module, in which each one of the at least one cold water circulating module has a cooling unit, a wrap, a connection hose assembly and a charging unit. The method has steps of:

(a) placing the cooling unit of each one of the at least one cold water circulating module of the wearable horse cooling device on a body of a horse;

(b) adjusting the fixing module and securely connecting the fixing module with each one of the at least one cold water circulating module;

(c) connecting the connection hose assembly with the cooling unit and the wrap of each one of the at least one cold water circulating module; and

(d) mounting the wrap of each one of the at least one cold water circulating module around a portion of the body of the horse; and

(e) setting operation parameters required for a cooling cycle.

Preferably, the fixing module has an adjustable neck strap and two adjustable girth straps; and the step (b) has steps of securely connecting the two adjustable girth straps to the cooling unit of each one of the at least one cold water circulating module and tightening the adjustable girth straps to fit the adjustable girth straps under the horse and firmly mounting the cooling unit of each one of the at least one cold water circulating module on the body of the horse, and securely connecting the adjustable neck strap to the cooling unit of each one of the at least one cold water circulating module and tightening the adjustable neck strap.

Preferably, the cooling unit has an ice container, a water pump, a pneumatic pump and an electronic controller, the charging unit has a battery set, and

the method further has a step of using the charging unit to charge the battery set and electrically connecting the battery set with the cooling unit of one of the at least one cold water circulating module before the step (a).

Preferably, the wrap has a water bag and an air bag, the connection hose assembly has an input water hose, an output water hose and an air hose;

the step (c) has steps of connecting the input water hose of the connection hose assembly between the water bag of the wrap and the ice container of the cooling unit of one of the at least one cold water circulating module and connecting the output water hose with the ice container, the water pump and the water bag of the cold water circulating module, connecting the air hose of the connection hose assembly between the air bag of the wrap and the pneumatic pump of the cooling unit of one of the at least one cold water circulating module, and filling in the ice container of the cooling unit of each one of the at least one cold water circulating module with ice cubes and clean water; and the step (d) has steps of inflating the air bag of the wrap of each one of the at least one cold water circulating module to hold the portion of the horse so as to mount the wrap around the portion of the horse, and fully filling in the water bag of the wrap of each one of the at least one cold water circulating module with cold water circulated from the ice container of the cold water circulating module.

Preferably, the operation parameters have a pressure value of the air bag serving for the wrap of each one of the at least one cold water circulating module to hold a portion of a horse and a time period of the cooling cycle.

The method for mounting a wearable horse cooling device has the advantages of firmly mounting the wearable horse cooling device on the back of a horse without causing discomfort of the horse, and allowing the horse carrying the wearable horse cooling device to freely move. Accordingly, the method for mounting a wearable horse cooling device solves the inconvenience of conventional horse cooling method during cooling operation.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a wearable horse cooling device in accordance with the present invention mounted on a house;

FIG. 2 is a perspective view of two cold water circulating modules of the wearable horse cooling device in FIG. 1 connected with each other by a connection strap;

FIG. 3 is a front view of an unfolded wrap of the wearable horse cooling device in FIG. 1;

FIG. 4 is an operational perspective view of the wearable horse cooling device in FIG. 1 mounted on a horse;

FIG. 5 is another operational perspective view of the wearable horse cooling device in FIG. 1 mounted on a horse;

FIG. 6 is a block diagram of a method for mounting a wearable horse cooling device in accordance with the present invention;

FIG. 7 is a block diagram of a step of adjusting the fixing module and securely connecting the fixing module in the method in FIG. 6;

FIG. 8 is a block diagram of a step of connecting the connection hose assembly in the method in FIG. 6; and

FIG. 9 is a block diagram of a step of mounting the wrap in the method in FIG. 6.

### DETAILED DESCRIPTION OF THE INVENTION

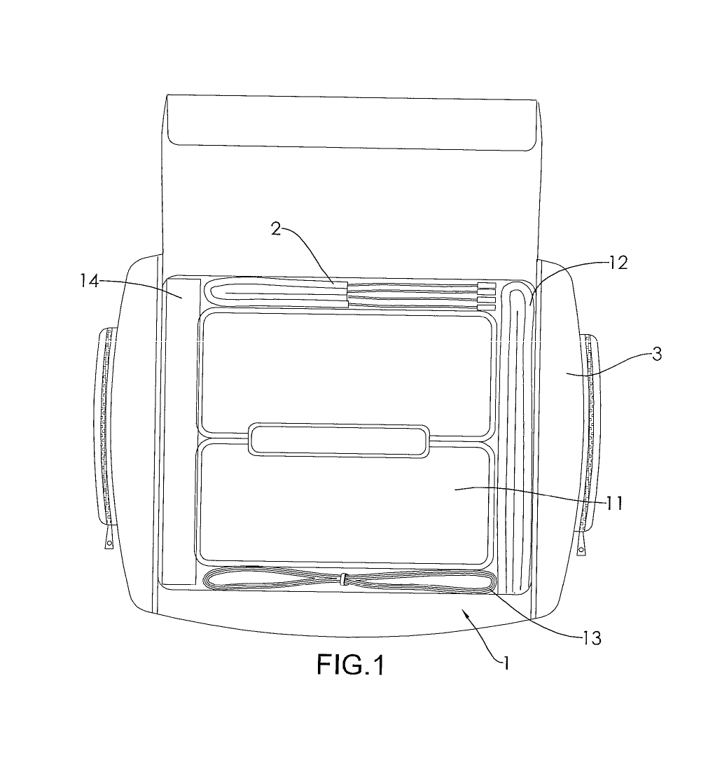

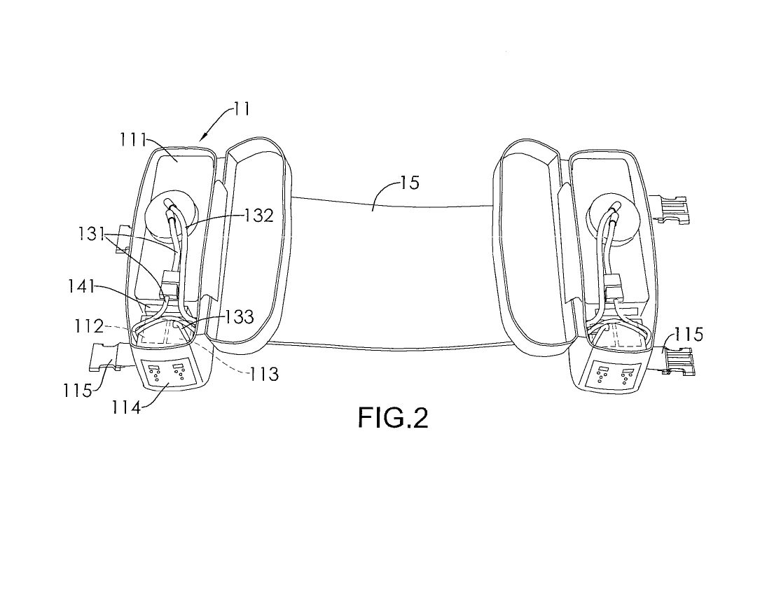

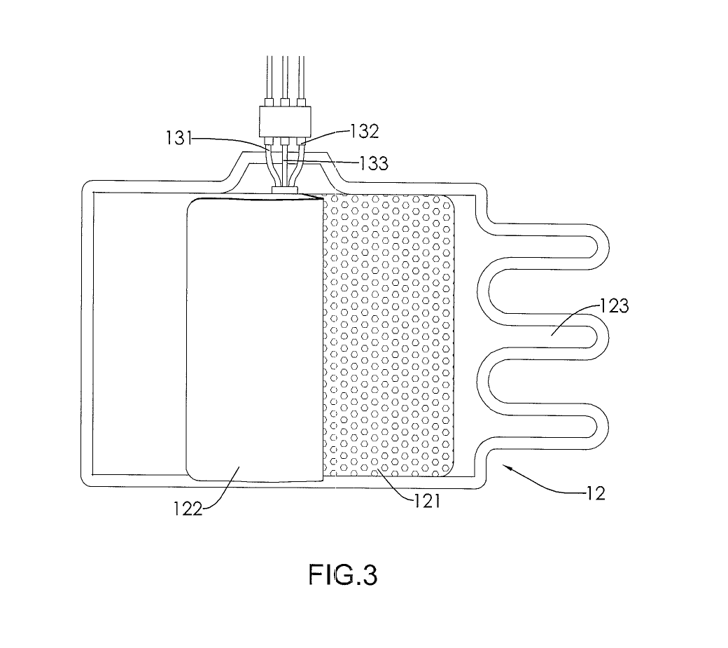

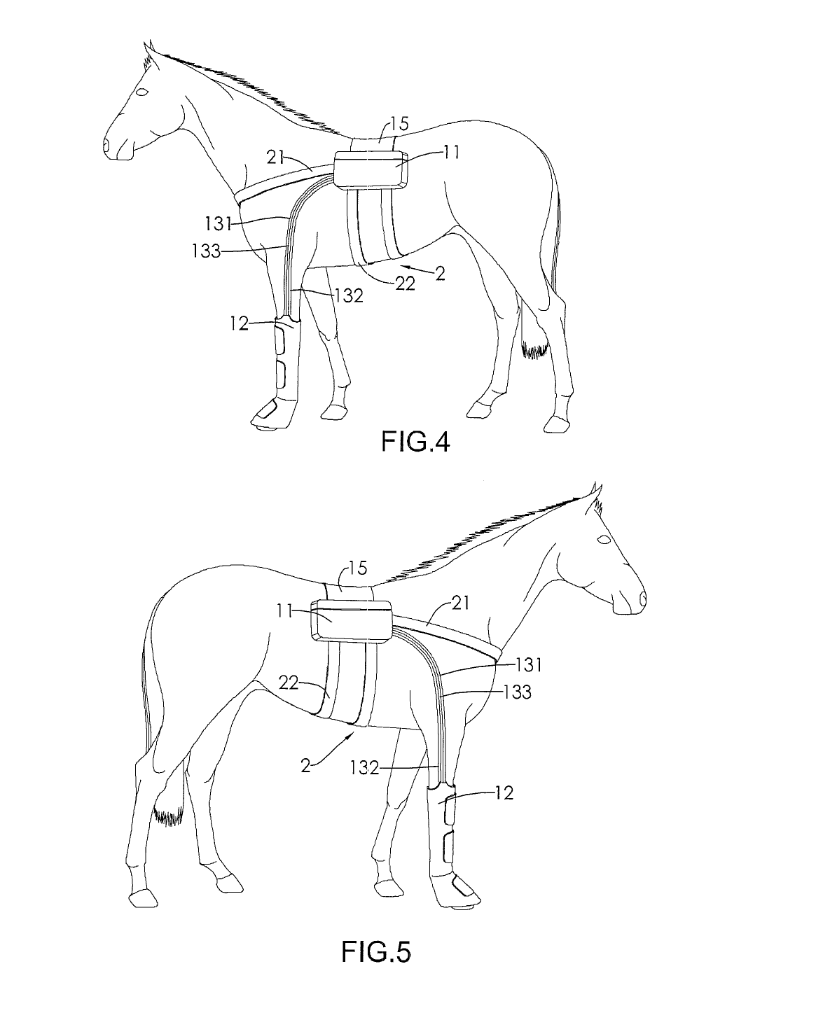

With reference to FIGS. 1 to 5, a wearable horse cooling device in accordance with the present invention has at least one cold water circulating module 1 and a fixing module 2. In the present embodiment, the wearable horse cooling device has two cold water circulating modules 1. Each cold water circulating module 1 has a cooling unit 11, a wrap 12, a connection hose assembly 13 and a charging unit 14. The cooling unit 11 has an ice container 111, a water pump 112, a pneumatic pump 113, and an electronic controller 114. The wrap 12 has a water bag 121, an air bag 122 and a fastener 123. The water bag 121 and the air bag 122 are mounted in the wrap 12. The air bag 122 has an automatic air release valve 124. The automatic air release valve 124 serves to automatically release the air overcharged in the air bag to prevent a wearing portion of the horse from being over-tightened or the air bag 122 from being blown up. The fastener 123 serves to be fastened around a portion of a horse. In the present embodiment, the wrap 12 is a leg wrap, and the fastener 123 is a Velcro fastener. The connection hose assembly 13 has an input water hose 131, an output water hose 132 and an air hose 133. The input water hose 131 and the output water hose 132 are connected between and communicates with the water pump 112 of the cooing unit 11 and the water bag 121 of the wrap 12 of one of the cold water circulating modules 1. The air hose 133 is connected between and communicates with the pneumatic pump 113 of the cooling unit 11 of each cold water circulating module 1 the and the air bag 122 of the wrap 12. The electronic controller 114 controls the water pump 112 to circulate cold water between the ice container 111 and the water bag 121 through the input water hose 131 and the output water hose 132 and controls the pneumatic pump 113 to inflate or deflate the air bag 122 through the air hose 133. When inflated or deflated, the air bag 122 holds or loosens a portion of a horse. The charging unit 14 is connected to the AC mains to charge a battery set 141 thereof for supplying an operating power to the wearable horse cooling device. The fixing module 2 is securely connected with the cold water circulating modules 1 and is tied to the body of a horse so as to firmly mount the cold water circulating unit 1 on the body of the horse without causing discomfort of the horse. In the present embodiment, the fixing module 2 has an adjustable neck strap 21 and two adjustable girth straps 22.

In the present embodiment, the two cooling units 11 of the two cold water circulating modules 1 are connected with each other by using a connection strap 15 mounted between the two cooling units 11 so that the two cooling units 11 can be mounted on two sides of a horse with the connection strap 15 striding across the horse back. The connection strap 15 is made of a soft and resilient material to ensure the horse's wearing comfort.

Each cooling unit 11 has two buckles 115 mounted on a bottom of the cooling unit 1. Each buckle 115 on one of the cooling units 11 can engage one end of one of the adjustable girth strap 22 of the fixing module 2 to facilitate fixing the cooling units 11.

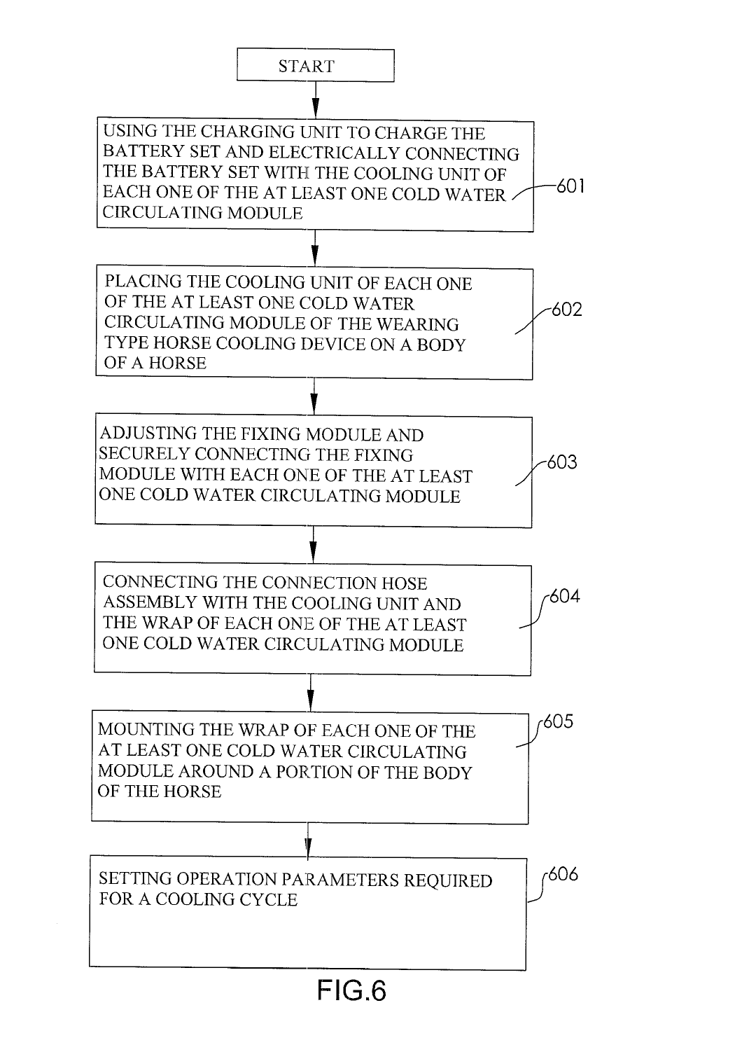

With reference to FIG. 6, a method for mounting a wearable horse cooling device in accordance with the present invention has the following steps of:

using the charging unit to charge the battery set and electrically connecting the battery set with the cooling unit of each one of the at least one cold water circulating module (601);

placing the cooling unit of each one of the at least one cold water circulating module of the wearable horse cooling device on a body of a horse (602);

adjusting the fixing module and securely connecting the fixing module with each one of the at least one cold water circulating module (603);

connecting the connection hose assembly with the cooling unit and the wrap of each one of the at least one cold water circulating module (604); and

mounting the wrap of each one of the at least one cold water circulating module around a portion of the body of the horse (605); and

setting operation parameters required for a cooling cycle (606).

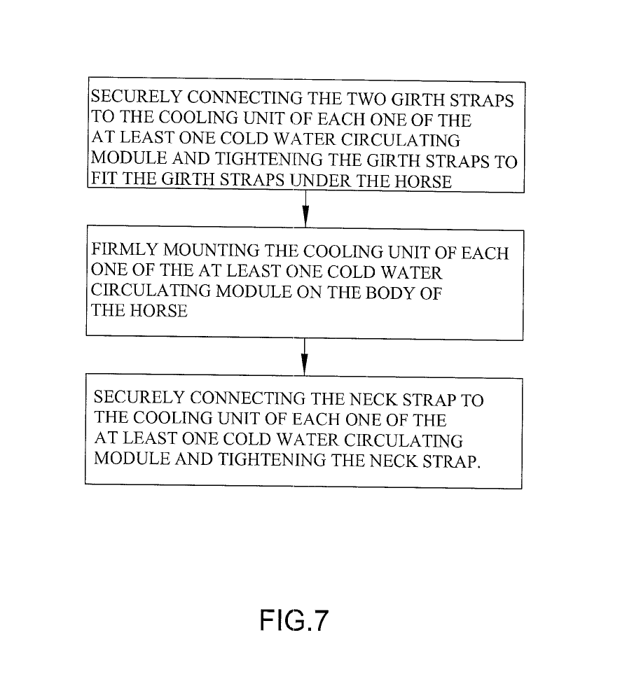

Considering that the connection hose assembly has one adjustable neck strap and two adjustable girth straps, with reference to FIG. 7, the step (603) has steps of:

securely connecting the two adjustable girth straps to the cooling unit of each one of the at least one cold water circulating module and tightening the adjustable girth straps to fit the adjustable girth straps under the horse;

firmly mounting the cooling unit of each one of the at least one cold water circulating module on the body of the horse; and

securely connecting the adjustable neck strap to the cooling unit of each one of the at least one cold water circulating module and tightening the adjustable neck strap.

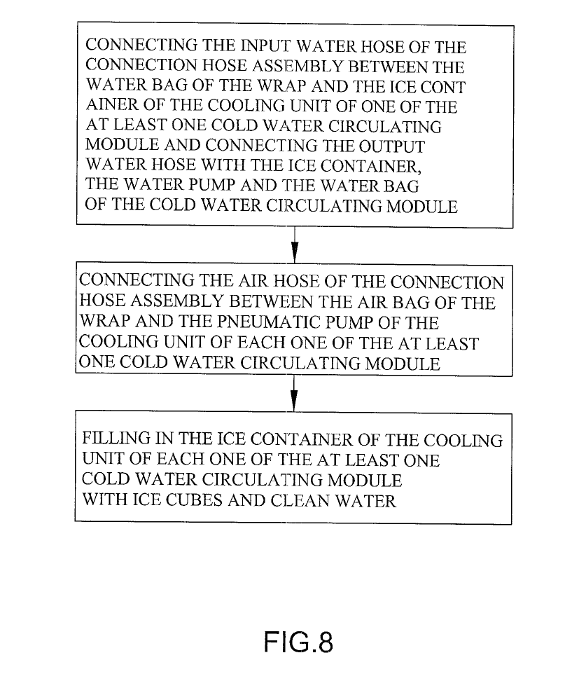

Considering that the wrap has a water bag and an air bag and the connection hose assembly has an input water hose, an output water hose and an air hose, with reference to FIG. 8, the step (604) has steps of:

connecting the input water hose of the connection hose assembly between the water bag of the wrap and the ice container of the cooling unit of one of the at least one cold water circulating module and connecting the output water hose with the ice container, the water pump and the water bag of the cold water circulating module;

connecting the air hose of the connection hose assembly between the air bag of the wrap and the pneumatic pump of the cooling unit of one of the at least one cold water circulating module; and

filling in the ice container of the cooling unit of each one of the at least one cold water circulating module with ice cubes and clean water.

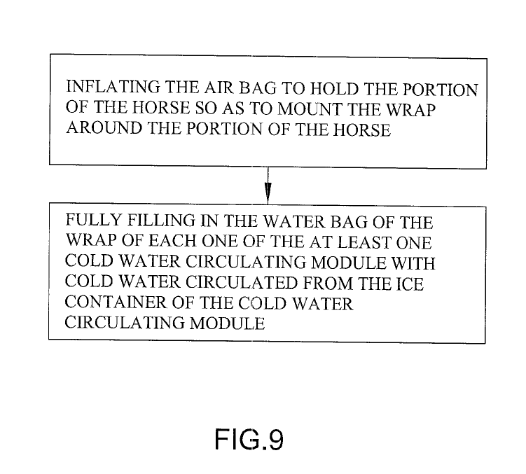

Considering that the wrap has a water bag and an air bag, with reference to FIG. 9, the step (605) has steps of:

inflating the air bag to hold the portion of the horse so as to mount the wrap around the portion of the horse; and

fully filling in the water bag of the wrap of each one of the at least one cold water circulating module with cold water circulated from the ice container of the cold water circulating module.

The operation parameters in step (606) have a pressure value of the air bag for the wrap of each one of the at least one cold water circulating module to hold a portion of a horse and a time period of the cooling cycle.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A method for mounting a wearable horse cooling device performed by a wearable horse cooling device having at least one cold water circulating module and a fixing module, wherein each one of the at least one cold water circulating module has a cooling unit, a wrap, a connection hose assembly and a charging unit, the method comprising steps of:
(a) placing the cooling unit of each one of the at least one cold water circulating module of the wearable horse cooling device on a body of a horse;
(b) adjusting the fixing module and securely connecting the fixing module with each one of the at least one cold water circulating module;
(c) connecting the connection hose assembly with the cooling unit and the wrap of each one of the at least one cold water circulating module; and
(d) mounting the wrap of each one of the at least one cold water circulating module around a portion of the body of the horse; and
(e) setting operation parameters required for a cooling cycle.

2. The method for mounting a wearable horse cooling device as claimed in claim 1, wherein
the fixing module has an adjustable neck strap and two adjustable girth straps; and
the step (b) has steps of:
securely connecting the two adjustable girth straps to the cooling unit of each one of the at least one cold water circulating module and tightening the adjustable girth straps to fit the adjustable girth straps under the horse;
firmly mounting the cooling unit of each one of the at least one cold water circulating module on the body of the horse; and
securely connecting the adjustable neck strap to the cooling unit of each one of the at least one cold water circulating module and tightening the adjustable neck strap.

3. The method for mounting a wearable horse cooling device as claimed in claim 2, wherein
the cooling unit has an ice container, a water pump, a pneumatic pump and an electronic controller;
the charging unit has a battery set; and
the method further comprises a step of using the charging unit to charge the battery set and electrically connecting the battery set with the cooling unit of one of the at least one cold water circulating module before the step (a).

4. The method for mounting a wearable horse cooling device as claimed in claim 3, wherein
the wrap has a water bag and an air bag;
the connection hose assembly has an input water hose, an output water hose and an air hose;
the step (c) has steps of:
connecting the input water hose of the connection hose assembly between the water bag of the wrap and the ice container of the cooling unit of one of the at least one cold water circulating module and connecting the output water hose with the ice container, the water pump and the water bag of the cold water circulating module;
connecting the air hose of the connection hose assembly between the air bag of the wrap and the pneumatic pump of the cooling unit of one of the at least one cold water circulating module; and
filling in the ice container of the cooling unit of each one of the at least one cold water circulating module with ice cubes and clean water; and
the step (d) has steps of:
inflating the air bag of the wrap of each one of the at least one cold water circulating module to hold the portion of the horse so as to mount the wrap around the portion of the horse; and
fully filling in the water bag of the wrap of each one of the at least one cold water circulating module with cold water circulated from the ice container of the cold water circulating module.

5. The method for mounting a wearable horse cooling device as claimed in claim 4, wherein
in the step (e), the operation parameters have:
a pressure value of the air bag for the wrap of each one of the at least one cold water circulating module to hold a portion of a horse; and
a time period of the cooling cycle.
